(19) United States
(12) Patent Application Publication
(10) Pub. No.: US 2013/0102060 A1
(43) Pub. Date: Apr. 25, 2013

(54) LANCE DEVICE AND ASSOCIATED METHODS FOR DELIVERING A BIOLOGICAL MATERIAL INTO A BIOLOGICAL STRUCTURE

(71) Applicant: NanoInjection Technologies, L.L.C., Salt Lake City, UT (US)

(72) Inventor: Quentin T. Aten, Orem, UT (US)

(73) Assignee: NANOINJECTION TECHNOLOGIES, L.L.C., Salt Lake City, UT (US)

(21) Appl. No.: 13/657,761

(22) Filed: Oct. 22, 2012

Related U.S. Application Data
(60) Provisional application No. 61/550,169, filed on Oct. 21, 2011.

(51) Int. Cl. C12M 1/00

(52) U.S. Cl. 435/285.1

(57) ABSTRACT

Systems, devices, and methods for delivering a biological material into a biological structure are provided. In one aspect, for example, a lance device for introducing biological material into a biological structure and configured for use in a nanoinjection system including a microscope is provided. Such a device can include a substrate including a handle region located between a manipulator coupling region and a lance shaft region, a lance tip operable to introduce biological material into a biological structure, the lance tip being coupled to the lance shaft region, and an electrically conductive layer extending from the manipulator coupling region to the lance tip, the conductive layer being configured to electrically couple to a power source. Thus, the conductive layer provides an electrical connection from the power source to the lance tip when in use.

### PRIORITY DATA

This application claims the benefit of U.S. Provisional Patent Application No. 61/550,169, filed on Oct. 21, 2011, which is incorporated herein by reference.

### BACKGROUND OF THE INVENTION

Microinjection of foreign materials into a biological structure such as a living cell can be challenging. Various transfection techniques include the microinjection of foreign genetic material such as DNA into the nucleus of a cell to facilitate the expression of foreign DNA. For example, when a fertilized oocyte (egg) is transfected, cells arising from that oocyte will carry the foreign genetic material. Thus in one application, organisms can be produced that exhibit additional, enhanced, or repressed genetic traits. In some cases, researchers have used microinjections to create strains of mice that carry a foreign genetic construct causing macrophages to auto-fluoresce and undergo cell death when exposed to a certain drugs. Such transgenic mice have since played roles in investigations of macrophage activity during immune responses and macrophage activity during tumor growth.

Prior art microinjectors function in a similar manner to macro-scale syringes: a pressure differential forces a liquid through a needle and into the cell. In some cases a glass needle that has been fire drawn from a capillary tube can be used to pierce the cellular and nuclear membranes of an oocyte. Precise pumps then cause the expulsion of minute amounts of genetic material from the needle and into the cell. Researchers have produced fine microinjection needles made from silicon nitride and silica glass that are smaller than fire drawn capillaries. These finer needles generally also employ macro-scale pumps similar to those used in traditional microinjectors.

### SUMMARY

The present disclosure provides systems, devices, and methods for delivering a biological material into a biological structure such as a cell. In one aspect, for example, a lance device for introducing biological material into a biological structure and configured for use in a nanoinjection system including a microscope is provided. Such a device can include a substrate including a handle region located between a manipulator coupling region and a lance shaft region, a lance tip operable to introduce biological material into a biological structure, the lance tip being coupled to the lance shaft region, and an electrically conductive layer extending from the manipulator coupling region to the lance tip, the conductive layer being configured to electrically couple to a power source. Thus, the conductive layer provides an electrical connection from the power source to the lance tip when in use. In another aspect, an insulating layer can be applied to the substrate to cover at least a portion of the conductive layer. In another aspect, the lance has a lance tip region that is configured to penetrate a biological membrane and a lance shaft region that is configured to support the lance tip region during penetration. In a further aspect, the conductive layer can be applied to the manipulator coupling region so as to form an electrical connection between the lance tip and the power source when the manipulator coupling region is engaged with a manipulator.

The support substrate can be comprised of a variety of materials, depending on the design intensions of the device. In one aspect, for example, the support substrate can be comprised of an electrically nonconductive material. In another aspect, the support substrate has an electrical resistance of at least 5000 ohm-cm. In a further aspect, the support substrate can be an electrically insulative material such as monosilicon.

In a further aspect, the lance device can be structurally configured to allow entry and movement of the lance tip into the biological structure along an elongate axis of the lance tip and along a focal plane of the microscope. In yet another aspect, the lance device is structurally configured to allow substantially horizontal entry and movement of the lance tip into the biological structure. In another aspect, the lance device can be structurally configured such that the lance tip remains in a focal plane of the microscope as the lance device is moved substantially horizontally into the biological structure along an elongate axis of the lance tip.

The present disclosure additionally provides nanoinjection systems for introducing biological material into a cell. In one aspect, for example, such a system can include a lance device having a substrate including a handle region located between a manipulator coupling region and a lance shaft region, a lance tip operable to introduce biological material into a biological structure, the lance tip being coupled to the lance shaft region, and an electrically conductive layer extending from the manipulator coupling region to the lance tip. The conductive layer can be configured to electrically couple to a power source and to provide an electrical connection from the power source to the lance tip when in use. Additionally, the lance device can be structurally configured to allow entry and movement of the lance tip into the cell along an elongate axis of the lance tip and along a focal plane of a viewing microscope. The system can further include a charging system electrically coupleable to the lance device and operable to charge and discharge the lance tip, and a lance device manipulation system coupleable to the lance device and operable to move the lance into and out of a cell in a reciprocating motion along an elongate axis of the lance tip.

In some aspects, the system can also include a microscope oriented such that a focal plane of the microscope is parallel to the elongate axis of the lance tip. In other aspects, the system can include a biological material delivery device configured to deliver a biological material capable of association with the lance tip. It can be beneficial in some aspects to position the biological material delivery device to deliver the biological material to contact the lance tip.

### BRIEF DESCRIPTION OF THE DRAWINGS

FIG. 1 is a schematic representation of various steps of the delivery of a biological material into a cell in accordance with one embodiment of the present invention.

FIG. 2a is a graphical representation of a lance device in accordance with another embodiment of the present invention.

FIG. 2b is a graphical representation of a lance device in accordance with another embodiment of the present invention.

FIG. 3a is a graphical representation of a lance device in accordance with another embodiment of the present invention.

FIG. 3b is a graphical representation of a portion of the lance device of FIG. 3a in accordance with another embodiment of the present invention.

FIG. 4a is a graphical representation of a lance system in accordance with another embodiment of the present invention.

FIG. 4b is a graphical representation of a portion of the lance system of FIG. 4a in accordance with another embodiment of the present invention.

### DEFINITIONS OF TERMS

In describing and claiming the present invention, the following terminology will be used in accordance with the definitions set forth below.

The singular forms “a,” “an,” and, “the” can include plural referents unless the context clearly dictates otherwise. Thus, for example, reference to “a support” can include reference to one or more of such supports, and reference to “an oocyte” can include reference to one or more of such oocytes.

As used herein, the term “biological structure” can include any structure having a biological origin. Non-limiting examples of such biological structures include cells, oocytes, zygotes, embryos, cellular tissue, and the like. Additionally, a biological structure can include subcomponents of cells, embryos, and tissues, such as for example, cellular organelles.

As used herein, the term “biological material” can refer to any material that has a biological use and can be delivered into a biological structure. As such, “biological material” can refer to materials that may or may not have a biological origin. Thus, such material can include natural and synthetic materials, as well as chemical compounds, dyes, and the like.

As used herein, the term “charged biological material” may be used to refer to any biological material that is capable of being attracted to or associated with an electrically charged structure. Accordingly, the term charged biological material may be used to refer to those molecules having a net charge, as well as those molecules that have a net neutral charge but possess a charge distribution that allows attraction to the structure.

As used herein, “associate” is used to describe biological material that is in electrostatic contact with a structure due to attraction of opposite charges. For example, DNA that has been attracted to a structure by a positive charge is said to be associated or electrically associated with the structure.

As used herein, the term “uncharged” when used in reference to a lance may be used to refer to the relative level of charge in the lance as compared to a charged biological material. In other words, a lance may be considered to be “uncharged” as long as the amount of charge on the needle structure is insufficient to associate therewith a useable portion of the charged biological material. Naturally, what is a useable portion may vary depending on the intended use of the biological material, and it should be understood that one of ordinary skill in the art would be aware of what a useable portion is given such an intended use. Additionally it should be noted that a lance with no measurable charge would be considered “uncharged” according to the present definition.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint without affecting the desired result.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

This same principle applies to ranges reciting only one numerical value as a minimum or a maximum. Furthermore, such an interpretation should apply regardless of the breadth of the range or the characteristics being described.

### DETAILED DESCRIPTION

The present disclosure provides methods, devices, and associated systems for delivering a biological material into a biological structure. As one non-limiting example, DNA can be delivered into a biological structure such as a cell or an organelle of a cell (e.g. a nucleus or a pronucleus), resulting in genomic integration of the DNA. In one aspect, biological material can be introduced into the cell using a delivery device, such as a lance, having an outer shape that is smaller than delivery devices that have previously been used, such as for example, hollow micropipettes. A lance having a smaller outer shape may be less disruptive to cellular structures, and thus may allow delivery of the biological material into a cell with less cellular damage.

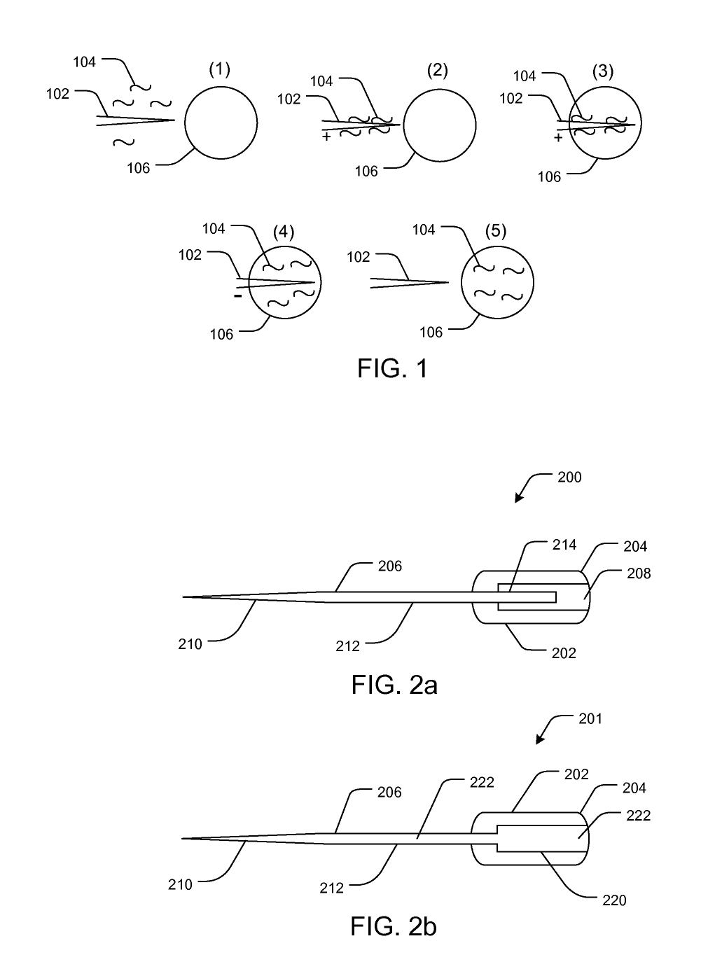

Various non-limiting steps can be performed to introduce biological material into a biological structure, as is described in the following sequence of actions. For this particular example, DNA is used as the biological material and the biological structure is a cell such as an oocyte. This example is intended to be non-limiting, and the description should be applied to other biological materials, cells, organelles, and the like. As is shown in FIG. 1 at (1), a lance 102 and DNA 104 are brought into proximity outside of a cell 106. The lance 102 is positively charged and brought into contact with the DNA 104 to cause the DNA 104 to be electrostatically accumulated at a tip region of the lance 102 as is shown at (2). The positive charge on the lance 102 thus causes the negatively charged DNA 104 to associate with and accumulate at the tip region. A return electrode can be placed in electrical contact with the medium surrounding the lance in order to complete an electrical circuit with the charging device (not shown). As is shown at (3), the lance 102 is inserted through the cell membrane and into the cell 106. DNA 104 associated with the tip portion is inserted into the cell 106 along with the lance 102. It is also contemplated that other techniques of associating the biological material with the lance in addition to electrostatic association are considered to be within the present scope. The DNA 104 is then released from the lance 102 within the cell 106, as is shown at (4). In the case of a charged lance, the lance 102 can be discharged to allow the release of at least a portion of the DNA 104, which is thus delivered into the cell. Discharge can be a reduction in charge (in this case positive charge) sufficient to release the DNA, or discharge can be a reversal in the polarity of the charge on the lance (e.g. negative charge). Following release of the DNA 104, the lance 102 can be withdrawn from the cell 106 as is shown at (5).

Because biological material can be loaded onto a lance and subsequently released via changes in the charge state of the lance, internal fluid delivery microinjection channels are not required for biological material delivery. As such, a lance can be smaller in size and can be formed in configurations that may not be possible with prior delivery devices.

It is contemplated that in some aspects the lance devices of the present disclosure can be utilized in traditional injection systems and setups with little modification. In other aspects, such lance devices can be utilized in dedicated nanoinjection systems designed for a specific type of lance or lance configuration. As such, it is understood that the design of the lance can vary depending on the system into which the lance is incorporated, and that specific design aspects can be present in every design regardless of the lance configuration.

Accordingly, in one aspect a lance device for introducing biological material into a biological structure provided. Such a lance can be configured for use in a nanoinjection system, which can include a microscope. As is shown in FIGS. 2a-b, the lance device 200 can include a handle region 202 having a manipulator coupling region 204 and a lance 206 capable of introducing biological material into a biological structure. The lance 206 is coupled to the handle region 202 in such a manner that the lance 206 is at least partially supported by the handle region 202. The lance device 200 can further include an electrical coupling pathway 208 configured to electrically couple to a power source (not shown). The electrical coupling pathway 208 provides an electrical connection from power source to the lance 206 when in use. As such, the electrical coupling pathway 208 makes electrical contact 214 with the lance 206. In the specific case of the aspect of FIG. 2a, the lance 206 is a conductive material, and thus the electrical coupling pathway 208 merely needs to electrically couple to a portion of the lance tip region 210 of the lance.

The configuration of the lance 206 can vary depending on the intended usage thereof. Any lance configuration is thus considered to be within the present scope. In one aspect, for example, the lance 206 can be a substantially straight and uniform structure. In another aspect, the lance 206 can have a lance tip region 210 configured to penetrate a biological membrane and a lance shaft region 212 configured to support the lance tip region 210. The lance 206 can thus be charged via the electrical coupling pathway 208 to attract a biological material to the lance tip region 210. The lance tip region 210 can then be inserted into a biological structure along with the associated biological material. Discharging or reversing the electrical charge on the lance 206 thus releases the biological material into the cell, following which the lance 206 can be withdrawn.

FIG. 2b shows a lance device 201 having a lance 206 in electrical connection with an electrical coupling pathway 222. In one aspect, the electrical conducting pathway 222 and the lance 206 can be a continuous material. In other words, the lance 206 and the electrical conductive pathway 222 can be made from the same conductive material. In another aspect, the electrical conductive pathway 222 can be a continuous or semi-continuous electrically conductive layer incorporated into the lance 206. As such, it is understood that a variety of configurations and designs can provide electrical coupling from one end of the lance to the other, and that any such configurations or designs are considered to be within the present scope.

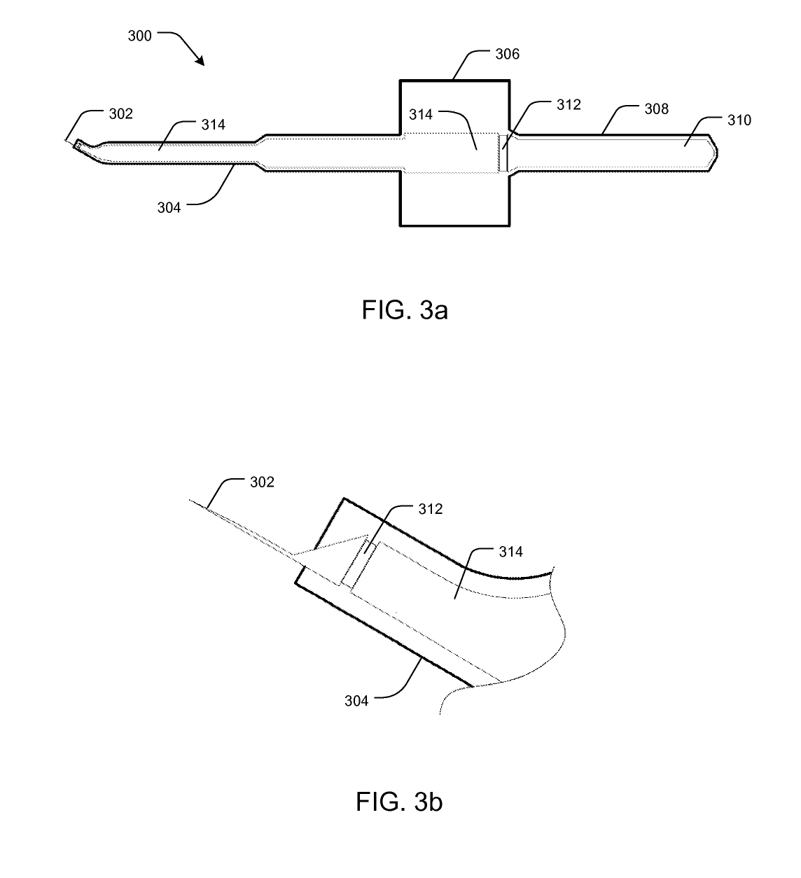

In another aspect, a lance device can be designed to readily engage and couple with a micromanipulator. In other words, the micromanipulator and the lance can be designed so that an electrical connection is formed by the act of coupling the lance device to the micromanipulator. As is shown in FIG. 3, for example, one exemplary design of a lance 300 can include a lance tip 302 supported by a lance support shaft 304. A handle region 306 provides sufficient structure to allow the lance device 300 to be grasped and manipulated by a user. An electrical and mechanical connecting shaft 308 or region extends from the handle region 306, and thus provides a connection structure to allow coupling with a micromanipulator or other manipulation device. The physical shape and configuration of the mechanical connecting shaft 308 can vary depending on the manipulator to which it is intended to couple. In the case of FIG. 3, the mechanical connecting shaft 308 is a male connection designed to engage and couple a corresponding female connection of the manipulator. An electrically conductive region 310 on the mechanical connecting shaft 308 thus contacts an electrical connection within the mating socket upon coupling.

In one aspect, the substrate including the lance support shaft 304, the handle region 306, and the mechanical connecting shaft 308 can be a continuous support substrate. A conductive layer 312 can be deposited on the handle region 306 and the lance support shaft 304 to provide electrical connectivity from the electrically conductive region 310 to the lance tip 302. This conductive layer is electrically coupled to the lance tip, thus allowing electrical charging of the lance. While the conductive layer 312 is shown as a distinct layer from the conductive region 310 in FIG. 3a, in some aspects a single continuous conductive layer can be utilized. As such, it is contemplated that the conductive layer 312 can be configured to complete an electrical pathway with a power source. For example, the mechanical connecting shaft 308 can engage with and couple to a female connector having a corresponding conductive layer that forms an electrical connection with the male connector when engaged. Coupling the lance device to the manipulator thus forms an electrical pathway from a power source to the lance tip. In an alternative embodiment, it is also contemplated that the lance device can include a female connector configured to mechanically and electrically couple to a male connector on the manipulator. It should thus be noted that the lance device should not be limited to the designs shown herein. For example, the lance, lance tip, lance support shaft, handling region, and mechanical and electrical connecting region can be designed in numerous ways, and with numerous materials, and still function as has been described.

Additionally, it can be beneficial to electrically isolate the conductive layer 312 from the local environment. In such cases, an insulating layer 314 can be applied over the conductive layer 312. The insulating layer 314 can be applied to any portion of the lance to insulate the conductive layer 312, including portions of the lance tip 302. Further details of the lance tip region are shown in FIG. 3b. In this case, the insulating layer 314 covers the conductive layer 312 up to the lance tip 302.

Thus, it is contemplated that a lance device can be fabricated and utilized in traditional manipulation systems such as micromanipulators and the like. Such manipulation systems will be referred to herein as lance manipulation systems or manipulators. As such, in some aspects the lance is manufactured as a “stand alone” lance, and is not constrained to a fixed substrate upon which the lance was fabricated. As one example, a lance can be manufactured from a precursor material, separated from that material, and coupled to a lance manipulation system. In addition to the lance itself, a coupling mechanism may be beneficial in order to couple the lance to a micromanipulator. In such cases the lance can be easily replaced with minimal effort. Furthermore, a charging system and a return electrode can be electrically coupled to the lance to facilitate lance charging and discharging.

Any size and/or shape of a lance or a lance tip capable of delivering biological material into a biological structure is considered to be within the present scope. The size and shape of the lance tip can also vary depending on the structure receiving the biological material. The effective diameter of the lance tip, for example, can be sized to maximize survivability of a cell. It should be noted that the term “diameter” is used loosely, as in some cases the cross section of the lance tip may not be circular. Limits on the minimum diameter of the lance tip can, in some cases, be a factor of the material from which the lance is made and the manufacturing process used. In one aspect, for example, the lance can have a tip diameter of from about 5 nm to about 3 microns. In another aspect, the lance can have a tip diameter of from about 10 nm to about 2 microns. In another aspect, the lance can have a tip diameter of from about 30 nm to about 1 micron. In a further aspect, the lance can have a tip diameter that is less than or equal to 1 micron. As such, in many cases the tip diameter of the lance can be smaller than the resolving power of current optical microscopes, which is approximately 1 micron.

The delivery of a biological material into a biological structure such as a cell is facilitated by high optical magnification due to the small sizes of such cells. Traditional optical microscopes having sufficient magnification for such delivery are generally oriented with an optical axis in a vertical direction, either coming from above the cell or below the cell for an inverted microscope. When a micropipette (or other delivery device) is inserted into a cell, the micropipette is generally directed toward the cell along an axis that does not correspond to a focal plane of the microscope. A focal plane of the microscope would be perpendicular to the optical axis. If the optical axis is thus oriented in a vertical direction, the focal plane would thus be oriented in a horizontal direction. Because the optics of the microscope are focused at the focal plane and the lance is not oriented along the focal plane, conventional systems have typically required that the lance be continually aligned both horizontally and vertically as it descends toward the cell. Additionally, to facilitate alignment, the microscope is often focused on the tip of the lance, and as such, must be refocused as the lance descends toward the cell and out of the current focal plane.

For some designs, the present disclosure provides the advantage of orienting the lance such that it may remain in the focal plane of the microscope as the lance is moved toward the cell. Many manipulation systems preclude such an orientation of the lance due to the proximity of the cell to an underlying substrate and the bulky nature of traditional micromanipulators. The size and physical configurations of the lances according to aspects of the present disclosure, however, allow such in-plane orientation of the lance. As such, in one aspect the present disclosure provides a lance for introducing biological material into a cell and configured for use in a nanoinjection system including a microscope. Such a lance can have a tip region and a shaft region, where the lance is configured to allow entry and movement of the tip region into the cell along an elongate axis of the tip region and along a focal plane of the microscope. It should be noted that the shape and overall configuration of the lance devices as described and shown in all of the figures should not be seen as limiting. It should also be noted that the present scope is not limited to lance device designs that remain in the focal plane as the lance is moved toward the cell, but also includes situations whereby the lance moves out of the focal plane as the manipulator is advanced.

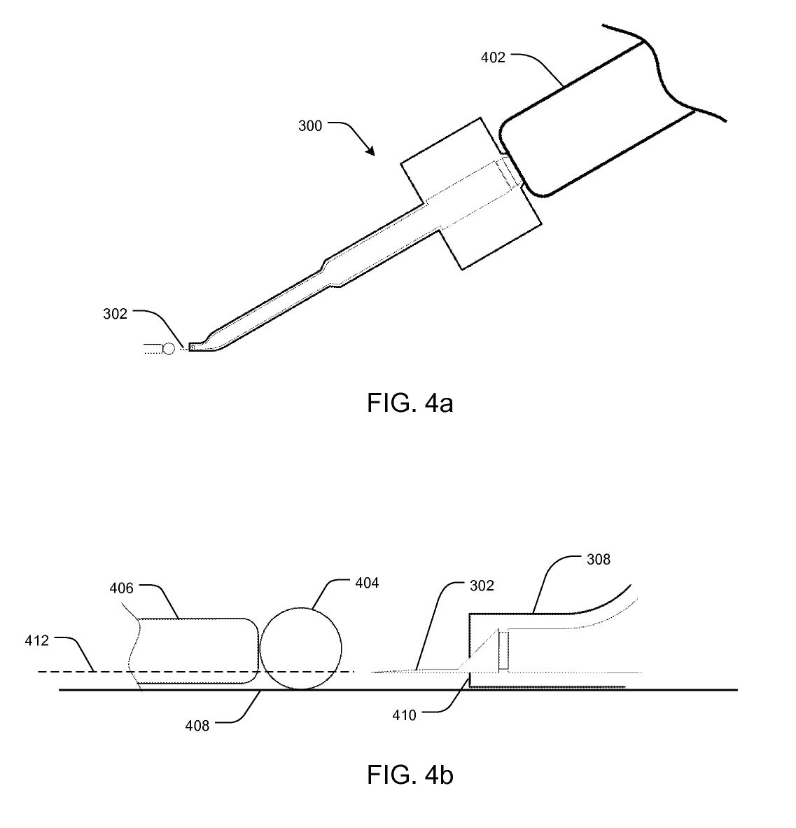

One example of such a configuration is shown in FIG. 4a. In this case, the lance device 300 of FIG. 3a is shown coupled to a micromanipulator coupling 402. As has been described, coupling the lance device 300 to the micromanipulator coupling 402 completes and electrical circuit between the micromanipulator coupling 402 and the lance tip 302. A larger depiction of this area of FIG. 4a is shown in FIG. 4b. The lance device 300 is oriented such that the lance tip 302 is adjacent to a cell 404 to be injected. The cell 404 is shown held by a suction pipette 406 and in close proximity to or touching a support substrate 408. Note that the lance support shaft 304 in this aspect has a lower portion 410 protruding below the lance tip 302. This lower portion can protect the lance tip 302 from contacting the support substrate 408 and being damaged.

A focal plane of the microscope is shown at 412. The lance tip 302 is thus substantially parallel to the focal plane 412, and will remain in focus as the lance tip 302 is moved substantially horizontally into the cell 404. The bent configuration of the lance device 300 can be beneficial to allow clearance between the micromanipulator and micromanipulator coupling 402 and the support substrate 408.

The lance tip 302 is shown having a substantially horizontal orientation that is in the focal plane 412. However, in various aspects it is contemplated that the focal plane and thus the tip portion of the lance can be in an orientational configuration that is not horizontal, but wherein the elongate axis of the tip portion of the lance is aligned within the focal plane. Thus, it is contemplated that that in some aspects the lance can be used at shallow angles for injections into a cell. In one aspect, for example, a shallow angle can be less than about 30° from the focal plane (or from horizontal). In another aspect, a shallow angle can be less than about 20° from the focal plane (or from horizontal). In yet another aspect, a shallow angle can be less than about 10° from the focal plane (or from horizontal). In a further aspect, a shallow angle can be less than about 5° from the focal plane (or from horizontal). In yet a further aspect, a shallow angle can be less than about 1° from the focal plane (or from horizontal).

The lance can be manipulated by any mechanism capable of aligning and moving the lance. Such a lance manipulation system can include any system or device capable of orienting and moving a lance. Non-limiting examples of lance manipulation systems include mechanical systems, magnetic systems, piezoelectric systems, electrostatic systems, thermo-mechanical systems, pneumatic systems, hydraulic systems, and the like. In one aspect, the lance manipulation system can be one or more micromanipulators. The lance may also be moved manually by a user. For example, a user may push the lance along a track from first location to a second location.

In one aspect, the lance can be moved by the lance manipulation system in a reciprocal motion along an elongate axis of the lance tip. In other words, the lance tip can move forward into a cell and backward out of the cell along the same path. By moving along the elongate axis of the lance tip, the minimum cross sectional area of the lance is driven through cellular structures such as a cell membrane and/or a cellular organelle. This minimal cross sectional exposure can limit the cellular disruption, and thus potentially increasing the success of the biological material delivery procedure.

A charging system used to charge the lance can include any system capable of electrically charging, maintaining the charge, and subsequently discharging the lance. Non-limiting examples can include batteries, DC power supplies, photovoltaic cells, static electricity generators, capacitors, and the like. The charging system can include a switch for activation and deactivation, and in some aspects can also include a polarity switch to reverse polarity of the charge on the lance. In one aspect the system may additionally include multiple charging systems, one system for charging the lance with a charge, and another charging system for charging the lance with an opposite polarity charge. In one example scenario, an initially uncharged lance is brought into contact with a sample of a biological material. The biological material can be in water, saline, or any other liquid capable of maintaining biological material. A charge opposite in polarity to the biological material is applied to the lance, thus associating a portion of the biological material with the lance. The lance can then be moved into the organelle of interest, and lance can be discharged, thus releasing the biological material.

The cell can be manipulated and or held in position by a variety of mechanisms. It should be noted that any technique, device, or system for manipulating and/or holding a cell in position is considered to be within the present scope. In one aspect, for example, the cell can be held in position by a suction pipette. A slight suction at the end of such a pipette can hold a cell for sufficient time to accomplish a biological material delivery procedure into an organelle of the cell. Additionally, supporting arms or other physically restraining structures can be used to hold the cell in position during the delivery procedure. Various configurations for support structures would be readily apparent to one of ordinary skill in the art once in possession of the present disclosure, and such configurations are considered to be within the present scope.

Further exemplary details regarding lances, charging systems, lance manipulation systems, and cellular restraining systems can be found in U.S. patent application Ser. Nos. 12/668,369, filed Sep. 2, 2010; 12/816,183; filed Jun. 15, 2010; 61/380,612, filed Sep. 7, 2010; and 61/479,777, filed on Apr. 27, 2011, each of which is incorporated herein by reference.

The length of the lance tip can be variable depending on the design and desired attachment of the lance to the lance manipulation system. Also, the portion of the lance tip that is contacting and/or passing through a portion of the cell can vary in length depending on the lance design and the depth of the area into which the biological material is to be delivered. For example, delivering biological material to an area located near the surface of a cell can be accomplished using a shorter lance tip as compared to delivery to an area located deep within the cell. This would not preclude, however, the use of longer lance tips for delivery into areas near the cellular surface. For example, a relatively long lance tip may be used to deliver biological material in an application where only a small portion (e.g., only the tip) of the lance tip penetrates a cell. It should be noted that the lance tip length can be tailored to the delivery situation and to the preference of the individual performing the delivery.

The overall shape and size of the lance tip can also be designed to take into account various factors, including those involved with the delivery procedure, as well as the materials utilized to make the lance. For example, in one aspect a lance can be designed having sufficient cross sectional strength to allow biological material delivery, while at the same time minimizing the damage done to the biological structure from the lance's cross sectional area. As another example, the lance can be designed to have a cross sectional area sufficient to minimize damage to the biological structure, while at the same having sufficient surface area to which biological material can be associated.

Different materials can also affect the design of the size and shape of the lance tip. Some materials may not hold a charge sufficient to associate the biological material to the lance tip at smaller sizes. In such cases, larger size lances can be used to facilitate a higher charge capacity. It may be difficult to form particular sizes and shapes of the lance from certain materials. In such cases, the lance size and shape can be designed to the properties of the desired material. For example, a material such as gold may not be capable of supporting the lance tip at very small diameters due to inadequate strength at smaller sizes, or it may not be possible or feasible to create a very small diameter tip with gold. If the use of a gold lance is desired, the lance size and shape can thus be designed with the properties of gold in mind.

As has been described, a charge is introduced into and held by the lance tip in order to electrically associate the biological material to the lance. Various lance tip materials are contemplated for use in constructing the lance, and any material that can be formed into a lance structure and is capable of carrying a charge is considered to be within the present scope. Non-limiting examples of such materials can include a metal or metal alloy, a conductive glass, a polymeric material, a semiconductor material, carbon nanotube, and the like, including combinations thereof. In one aspect, a lance tip can be a carbon nanotube filled with a material such as carbon, silicon, and the like. Non-limiting examples of metals can include indium, gold, platinum, silver, copper, palladium, tungsten, aluminum, titanium, and the like, including alloys and combinations thereof. Polymeric materials that can be used to construct the needle structure can include any conductive polymer, non-limiting examples of which include polypyrrole doped with dodecyl benzene sulfonate ions, SU-8 polymer with embedded metallic particles, and the like, including combinations thereof.

Non-limiting examples of useful semiconductor materials can include germanium, gallium arsenide, and silicon, including various forms of silicon such as amorphous silicon, monocrystalline silicon, polycrystalline silicon, and the like, including combinations thereof. Indium-tin oxide is a material that is also contemplated for use as a lance material. Additionally, in some aspects the support substrate and/or lance tip can be a conductive material that is coated on a second material, where the second material provides the physical structure of the lance. Examples can include metal-coated glass or metal-coated quartz lances. The lance can also include a hollow, non-conductive material, such as a glass, where the hollow material is filled with a conductive material. Depending on the design, the lance can be manufactured using various techniques such as wire pulling, chemical etching, MEMs processing, vapor deposition, sputtering, and the like.

It should be noted, that various materials begin to decompose (e.g. by electrolysis) at voltages above a certain threshold voltage referred to as the decomposition voltage. The decomposition voltage can be different for different materials. In some cases, such decomposition can generate oxygen and hydrogen at the positively charged lance and the negatively charged return electrode, respectively. These electrolysis products can cause damage to the lance tip and negatively affect the cell being injected. As such, in one aspect the voltage that can be used to charge the lance can be at or below the decomposition voltage. In one specific aspect, the lance tip is charged with a voltage from about 1 V below the decomposition voltage to about the decomposition voltage. In another aspect, the lance is charged with a voltage from about 2 V below the decomposition voltage to about the decomposition voltage.

Additionally, voltages higher than the decomposition voltage can cause the biological material to electrophoretically move to the lance. The higher the voltage, the more quickly the biological material will move to and associate with the lance. As such, in some aspects a charging voltage that is higher than the decomposition voltage of the lance can be used. In one aspect, for example, the lance is charged with a voltage from about the decomposition voltage to about 1 V above the decomposition voltage. In another aspect, the lance is charged with a voltage from about the decomposition voltage to about 2 V above the decomposition voltage. In yet another aspect, the lance is charged with a voltage from about the decomposition voltage to about 5 V above the decomposition voltage. In a further aspect, the lance is charged with a voltage that is greater than about 5 V above the decomposition voltage. Additionally, such charging can be described in terms that do not include decomposition voltage. In one aspect, for example, the lance is charged with a voltage from about 0.5 to about 5.0 V. In another aspect, the lance is charged with a voltage from about 1.0 V to about 3 V. In yet another aspect, the lance is charged with a voltage of about 1.5 V.

In one aspect, a lance tip and/or support substrate can be fabricated using MEMS processing from semiconductive or other MEMS capable materials. For example, a polysilicon lance tip can be made in conjunction with a silicon substrate (e.g. monosilicon), where the silicon substrate can be used to couple the lance to a lance manipulation system. The silicon substrate can electrically isolate the conductive layer from the environment, while a conductive layer can allow electrical connection between a charging system and the polysilicon lance via an electrically conductive pathway. Accordingly, very small lance tip sizes can be manufactured by using such processes, yet can still be easily handled due to the relatively large size of the substrate material. It should be noted that polysilicon and silicon are merely exemplary, and any material that can be formed into such a lance can be utilized.

The support substrate can be made from a variety of materials, and in some cases can be a bulk substrate material upon which the lance is formed or otherwise coupled. Any material capable of providing adequate support for the lance during handling and use is considered to be within the present scope. Non-limiting examples include metals, metal alloys, ceramics, polymeric materials, semiconductor materials, and the like, including combinations thereof. The handle region can also be electrically conductive to electrically non-conductive depending on the design of the device.

The design of a system for delivering biological material into a cell can vary due to the interdependencies of various system parameters. Combinations of features can thus influence other features, both in terms of system design and in terms of system use. Features can thus be mixed and matched to create a delivery system for a given purpose or desirable performance. For example, the materials and configuration chosen for the lance may have properties allowing a greater or lesser charge capacity, thus influencing the voltage, current, and electrical timing of the charging and discharging. A smaller tip diameter can more effectively enter an organelle with potentially less damage, but may have a smaller surface area for the association of biological material. The association capacity of the lance for biological material can thus be increased, for example, by utilizing lance materials capable of holding a higher relative charge, or by utilizing a non-circular shape for the lance tip that increases surface area while minimizing the penetration damage of the lance. Thus, if a particular feature is desired for a lance, other features can be varied to accommodate such a design. As such, it should be understood that the various details described herein should not be seen as limiting, particularly those involving dimensions or values. It is contemplated that a wide variety of design choices are possible, and each are considered to be within the present scope.

Furthermore, biological material can be delivered into a variety of biological structures such as cells and cellular structures. Both prokaryotic and eukaryotic cells are contemplated that can receive biological material, including cells derived from, without limitation, mammals, plants, insects, fish, birds, yeast, fungus, and the like. Additionally, cells can include somatic cells or germ line cells such as, for example, oocytes and zygotes. The enhanced survivability of cells with the present techniques can allow the use of cells and cell types that have previously been difficult to microinject due to their delicate nature. Additionally, biological structures can include any material of a biological origin, such as for example, embryos, cellular tissue, and the like.

Additionally, various types of biological materials are contemplated for delivery into a biological structure, and any type of biological material that can be electrostatically delivered is considered to be within the present scope. Non-limiting examples of such biological materials can include DNA, cDNA, RNA, siRNA, tRNA, mRNA, microRNA, peptides, synthetic compounds, polymers, dyes, chemical compounds, organic molecules, inorganic molecules, and the like, including combinations thereof. In one aspect, the biological material can include DNA, cDNA, RNA, siRNA, tRNA, mRNA, microRNA, and combinations thereof. In another aspect, the biological material can include DNA and/or cDNA.

It is to be understood that the above-described compositions and modes of application are only illustrative of preferred embodiments of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

1. A lance device for introducing biological material into a biological structure and configured for use in a nanoinjection system including a microscope, comprising:
a substrate including a handle region located between a manipulator coupling region and a lance shaft region;
a lance tip operable to introduce biological material into a biological structure, the lance tip being coupled to the lance shaft region; and
an electrically conductive layer extending from the manipulator coupling region to the lance tip, the conductive layer being configured to electrically couple to a power source, the conductive layer providing an electrical connection from the power source to the lance tip when in use.

2. The lance device of claim 1, further comprising an insulating layer applied to the substrate and covering at least a portion of the conductive layer.

3. The device of claim 1, wherein the lance has a lance tip region that is configured to penetrate a biological membrane and a lance shaft region that is configured to support the lance tip region during penetration.

4. The device of claim 1, wherein the conductive layer is applied to the manipulator coupling region so as to form an electrical connection between the lance tip and the power source when the manipulator coupling region is engaged with a manipulator.

5. The device of claim 1, wherein the support substrate is comprised of an electrically nonconductive material.

6. The device of claim 5, wherein the support substrate has an electrical resistance of at least 5000 ohm-cm.

7. The device of claim 6, wherein the support substrate is monosilicon.

8. The device of claim 1, wherein the lance device is structurally configured to allow entry and movement of the lance tip into the biological structure along an elongate axis of the lance tip and along a focal plane of the microscope.

9. The device of claim 1, wherein the lance device is structurally configured to allow substantially horizontal entry and movement of the lance tip into the biological structure.

10. The device of claim 8, wherein the lance device is structurally configured such that the lance tip remains in a focal plane of the microscope as the lance device is moved substantially horizontally into the biological structure along an elongate axis of the lance tip.

11. A nanoinjection system for introducing biological material into a cell, comprising:
a lance device including:
a substrate including a handle region located between a manipulator coupling region and a lance shaft region;
a lance tip operable to introduce biological material into a biological structure, the lance tip being coupled to the lance shaft region; and
an electrically conductive layer extending from the manipulator coupling region to the lance tip, the conductive layer being configured to electrically couple to a power source, the conductive layer providing an electrical connection from the power source to the lance tip when in use, wherein the lance device is structurally configured to allow entry and movement of the lance tip into the cell along an elongate axis of the lance tip and along a focal plane of a viewing microscope;
a charging system electrically coupleable to the lance device and operable to charge and discharge the lance tip; and
a lance device manipulation system coupleable to the lance device and operable to move the lance into and out of a cell in a reciprocating motion along an elongate axis of the lance tip.

12. The system of claim 11, further comprising a microscope oriented such that a focal plane of the microscope is parallel to the elongate axis of the lance tip.

13. The system of claim 11, further comprising a biological material delivery device configured to deliver a biological material capable of association with the lance tip.

14. The system of claim 11, wherein the biological material delivery device is positioned to deliver the biological material to contact the lance tip.
